(19) United States
(12) Patent Application Publication
(10) Pub. No.: US 2010/0055668 A1
(43) Pub. Date: Mar. 4, 2010

(54) Fluid-Transfer Collection Assembly Including Breakable Vial and Method of Using Same

(75) Inventor: David Karl Stroup, La Jolla, CA (US)

Correspondence Address: PROCOPIO, CORY, HARGREAVES & SAVITCH LLP, 530 B STREET, SUITE 2100, SAN DIEGO, CA, 92101, US

(73) Assignee: Infusion Innovations, Inc., La Jolla, CA (US)

(21) Appl. No.: 12/549,218

(22) Filed: Aug. 27, 2009

Related U.S. Application Data
(60) Provisional application No. 61/092,948, filed on Aug. 29, 2008.

(51) Int. Cl. C12Q 1/00; G01N 1/00; G01N 33/00; G01N 33/536

(52) U.S. Cl. 435/4; 436/174; 436/86; 436/94; 436/536; 422/61

(57) ABSTRACT

A method of using a fluid transfer and mixing collection assembly includes depressing a flexible member to cause a force to be imparted to a breakable vial to break the vial, releasing the second fluid therein into an interior of the flexible member; releasing the flexible member to impart a negative pressure in the interior of the flexible member to draw a first fluid into the interior of the flexible member through the inlet check valve to mix with the second fluid; and depressing the flexible member to impart a positive pressure in the interior of the flexible member to pump the mixed first fluid and second fluid out of the interior of the flexible member through an outlet check valve and be transferred to test media.

### CROSS-REFERENCE TO RELATED APPLICATION

This application claims the benefit of provisional patent application 61/092,948, filed Aug. 29, 2008 under 35 U.S.C. 119(e). This provisional patent application is incorporated by reference herein as though set forth in full.

### FIELD OF THE INVENTION

The present invention is, in general, in the field of fluid-transfer collection assemblies, and, in particular, in the field of fluid transfer and mixing collection assemblies.

### BACKGROUND OF THE INVENTION

Collection kits used for testing one or more analytes of a sample include multiple separate components such as a pipettes, collection tubes, vials or ampoules containing needed diluents or reagents, and test media devices. Because these collection kits have so many separate pieces, in most cases, use of such collection kits has been limited to a laboratory. Simple tests may be performed outside of the laboratory using only test media devices, but these test media devices are limited as to the types of tests that can be performed. More elaborate tests require diluents, pipettes, collection tubes, etc., and are difficult and awkward to perform outside of the laboratory.

Accordingly, a need exists for a simple fluid transfer and mixing collection assembly that does not include numerous separate pieces, is easy to use, can be used for multiple different types of tests and can be used in and outside a laboratory.

### SUMMARY OF INVENTION

Accordingly, an aspect of the invention involves a fluid transfer and mixing collection assembly. The collection assembly includes a base, a test media carried by the base, an inlet for receiving a first fluid, the inlet including an inlet check valve, an outlet including an outlet check valve, a glass vial carried by the base between the inlet and the outlet and including an interior with a second fluid therein, and a pair of depressable, flexible members carried on opposite sides of the base between the inlet and the outlet and including an interior with the glass vial disposed therein. The flexible members are depressable to break the glass vial therein, releasable to draw the first fluid into the interior of the flexible members through the inlet check valve to mix with the second fluid from the glass vial, and depressable again to pump the mixed first and second fluids out of the interior of the flexible members through the outlet check valve and be transferred to the test media.

Another aspect of the invention involves a method of using a fluid transfer and mixing collection assembly. The method includes providing a fluid transfer and mixing collection assembly including a base, a test media carried by the base, an inlet for receiving a first fluid, the inlet including an inlet check valve, an outlet including an outlet check valve, a breakable vial carried by the base between the inlet and the outlet and including an interior with a second fluid therein, and a depressable, flexible member carried by the base between the inlet and the outlet, the flexible member including an interior with the breakable vial therein; depressing the flexible member to cause a force to be imparted to the breakable vial to break the vial, releasing the second fluid therein into the interior of the flexible member; releasing the flexible member to impart a negative pressure in the interior of the flexible member to draw the first fluid into the interior of the flexible member through the inlet check valve to mix with the second fluid; and depressing the flexible member to impart a positive pressure in the interior of the flexible member to pump the mixed first fluid and second fluid out of the interior of the flexible member through the outlet check valve and be transferred to the test media.

A further aspect of the invention involves a fluid transfer and mixing collection assembly. The fluid transfer and mixing collection assembly includes a base; a test media carried by the base; an inlet for receiving a first fluid, the inlet including an inlet check valve; an outlet including an outlet check valve; a breakable vial carried by the base between the inlet and the outlet and including an interior with a second fluid therein, and a depressable, flexible member carried by the base between the inlet and the outlet, the flexible member including an interior with the breakable vial therein, the flexible member depressable to cause a force to be imparted to the breakable vial to break the vial, releasing the second fluid therein into the interior of the flexible member, the flexible member releasable to impart a negative pressure in the interior of the flexible member to draw the first fluid into the interior of the flexible member through the inlet check valve to mix with the second fluid, and the flexible member depressable again to impart a positive pressure in the interior of the flexible member to pump the mixed first fluid and second fluid out of the interior of the flexible member through the outlet check valve and be transferred to the test media.

Other and further objects, features, aspects, and advantages of the present inventions will become better understood with the following detailed description of the accompanying drawings.

### BRIEF DESCRIPTION OF THE DRAWINGS

FIG. 1 is a perspective view of a fluid transfer and mixing collection assembly constructed in accordance with an embodiment of the invention.

FIG. 2 is a cut-away perspective view of the fluid transfer and mixing collection assembly.

### DESCRIPTION OF THE PREFERRED EMBODIMENT

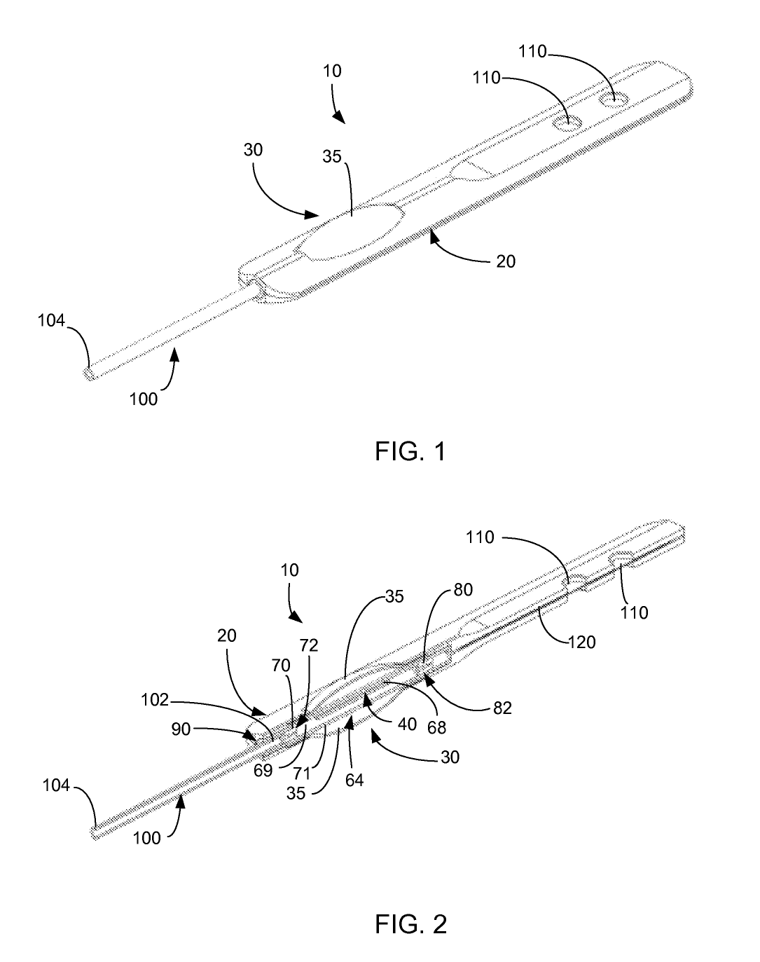

With reference to FIGS. 1 and 2, an embodiment of a fluid transfer and mixing collection assembly 10, and method of using the same will now be described. Further below, the collection assembly 10 will be described as an optical assay test device in an optical assay test method; however, the collection assembly 10 may be used in other devices, processes, and applications where mixing of two or more fluids and/or delivery of one or more fluids to a collection area is desired.

The collection assembly 10 includes a substantially flat, rectangular, elongated, plastic base 20 that carries a bulb pump 30 comprised of a pair of depressable, flexible, domed elastic members 35 carried on opposite sides of the base 20 and a glass vial 40 disposed in an interior of the bulb pump 30/members 35.

Although the bulb pump 30 is shown as being a pair of depressable members located on an upper surface and a bottom surface of the base 20, and oriented outwardy, away from the base 20, in alternative embodiments, the bulb pump 30 may be a single depressable member, may be located at other locations on the base 20, and/or may be oriented in one or more of an upward, a downward, a lateral, a forward, and a rearward direction with respect to the base 20. Similarly, the glass vial 40 may be located at other locations on the base 20, oriented differently, and/or fixed/mounted differently. In the embodiment shown, the glass vial has an elongated, narrow, cylindrical configuration. In alternative embodiments, breakable vessels made of other materials (e.g., plastic) and/or having other configurations may be used.

In the embodiment shown, fluid in an interior 64 of bulb pump 30 is air and fluid in an interior 68 of the vial 40 is one or more chemical reagents or diluents. In alternative embodiments, one or more different types of fluids may be used in the bulb pump 30 and/or the vial 40.

The interior 64 of the bulb pump 30 forms a fluid path 69 (formed by interior walls 71) between an inlet check valve 70 of an inlet 72 and an outlet check valve 80 of an outlet 82. The inlet 72 may include an inlet port 90 that communicates with a sample tube 100. The sample tube 100 may include a proximal end 102 and a distal end 104. The outlet check valve 80 communicates with one or more test media 110 via one or more fluid paths 120. One or more vents (not shown) in the collection assembly 10 allow air to escape where needed so that the pumping and fluid transferring action can be performed.

In the embodiment shown, the one or more test media 110 may include visual indicia to visually indicate the presence, absence, or concentration of a target analyte or other target object(s). The test media 110 may include one or more of the following: base strip(s), sample pad(s), conjugate pad(s), membrane(s), and absorbent pad(s).

The collection assembly 10 will now be described in use as an optical assay test device in an exemplary optical assay method of use. The collection assembly 10 and method of use may be used in applications such as, but not by way of limitation, drug screening, chemical analysis, crime/accident scene investigations, ground water testing (EPA), and livestock testing. In alternative embodiments, the collection assembly 10 is used in other fluid transfer and/or fluid collection applications.

The distal end 104 of the sample tube 100 may be put in communication with a fluid sample source. The fluid sample may be any fluid medium such as, but not by way of limitation, a gas, a liquid, a suspension, an extracted or dissolved sample, or a supercritical fluid, as long as some flow properties exist in the sample. The sample may include one or more target analytes of interest for detection. Example analytes include, but not by way of limitation, antigens, antibodies, receptors, ligands, chelates, proteins, enzymes, nucleic acids, DNA, RNA, pesticides, herbicides, inorganic or organic compounds or any material for which a specific binding reagent may be found.

The bulb 30 is depressed by squeezing the opposite flexible, domed elastic members 35 inwardly, towards each other. As the opposite flexible, domed elastic members 35 are pressed inwardly, the glass vial 40 deforms and, then, breaks, causing fluid therein to be released/flow out of the vial 40.

Release of the bulb 30 creates a vacuum force in the bulb 30, causing the sample to flow from the fluid sample source, through the tube 100 and the inlet check valve 70, into the fluid path 69 in the interior 64 of the bulb 30, where the sample mixes and reacts with the reagent released from the vial 40.

The bulb 30 is depressed again, causing the resulting reaction fluid to flow via the fluid path 69 out of the interior 64 of the bulb 30 and broken vial 40, through the outlet check valve 80 and the one or more fluid paths 120, and to the one or more test media 110. The visual indicia of the one or more test media 110 may indicate the presence, absence, or concentration of a target analyte for the optical assay method. In an embodiment of the invention, multiple test media 110 are used to test for the presence, absence, or concentration of a target analyte of interest.

Numerous features, implementations, and embodiments of the collection assembly 10 will now be described. The collection assembly 10 may be used more than once to perform the same test, different tests, or may be disposed of after single use. Different collection assemblies 10 may be used to perform different tests. The collection assembly 10 may be used to test for the presence, absence, or concentration of one or more analytes. The collection assembly 10 may be held and operated with a single hand of a user. The user may operate the depressable members 35 of the bulb pump 30 with a thumb and second digit of the same hand used to hold the collection assembly 10. In an alternative embodiment, the collection assembly 10 may have only one depressable member 35. The collection assembly 10 is especially advantageous in that the multiple transfer and mixing steps can all be done with a single hand of the user.

Although the embodiment of the collection assembly 10 shown includes a single glass vial 40 with one or more chemical reagents or diluents, in an alternative embodiment, the collection assembly 10 may have multiple breakable viles or vessels 40. The viles 40 may contain the same or different reagent(s)/diluent(s). Further, the collection assembly 10 may have one or more viles 40 containing one or more reagent(s)/diluent(s) and/or one or more separate reagent(s)/diluent(s) may be used with collection assembly 10 during the test process.

In one or more embodiments of the collection assembly 10, the sample tube 100 may have one or more of the following: the sample tube 100 may be fixed to the inlet 72, the sample tube 100 may be retractable, the sample tube 100 may not be retractable, the sample tube 100 may lock to the inlet 72, the sample tube 100 may not lock 72 to the inlet 72, the sample tube 100 may detachably connectable to the inlet 72, the sample tube 100 may include or be replaced with one or more wicks, sponges, open-cell foams, porous materials, or other absorbent materials.

In a further embodiment, the collection assembly 10 may include one or both of the inlet check valve 70 and the outlet check valve 80. Further, one or both of the inlet check valve 70 and the outlet check valve 80 may be replaced with one or more different types of valves. Still further, the collection assembly 10 may have a number of valves other than that shown, the number of valves depending on the number of bulb pumps 30.

The assembly 10 is advantageous in that it can be gripped in one hand and by the simple action of pressing and releasing the depressable members 35 of the bulb pump 30 with two digits of the same hand, fluid can be drawn into the fluid path 69 of the interior 64 of the bulb pump 30 through the inlet check valve 70. Pressing and releasing the bulb pump 30 can cause the vial 40 to break (so the fluid therein is released) and the fluids to mix in the interior 64 of the bulb pump 30. Pressing the bulb pump 30 again pumps the fluid out of the bulb pump 30 through the outlet check valve 80. In an exemplary embodiment of the assembly 10, the fluid pumped out of the bulb pump 30 can be collected on one or more test media to test the fluid for the presence, absence, or concentration of a target object in the fluid. Because the unit is so simple to use, the assembly 10 may be used by the user for testing in the field, in the lab, and in the home for a wide variety of applications.

The above figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

We claim:

1. A method of using a fluid transfer and mixing collection assembly, comprising:
providing a fluid transfer and mixing collection assembly including a base, a test media carried by the base, an inlet for receiving a first fluid, the inlet including an inlet check valve, an outlet including an outlet check valve, a breakable vial carried by the base between the inlet and the outlet and including an interior with a second fluid therein, and a depressable, flexible member carried by the base between the inlet and the outlet, the flexible member including an interior with the breakable vial therein;
depressing the flexible member to cause a force to be imparted to the breakable vial to break the vial, releasing the second fluid therein into the interior of the flexible member;
releasing the flexible member to impart a negative pressure in the interior of the flexible member to draw the first fluid into the interior of the flexible member through the inlet check valve to mix with the second fluid;
depressing the flexible member to impart a positive pressure in the interior of the flexible member to pump the mixed first fluid and second fluid out of the interior of the flexible member through the outlet check valve and be transferred to the test media.

2. The method of claim 1, wherein the vial is a glass vial, and depressing the flexible member includes depressing the flexible member to cause a force to be imparted to the breakable vial to break the vial, releasing the second fluid therein into the interior of the flexible member.

3. The method of claim 2, wherein the glass vial is fixably mounted within the interior of the flexible member, and depressing the flexible member includes depressing the flexible member to cause a force to be imparted to the breakable vial fixably mounted within the interior of the flexible member to break the vial, releasing the second fluid therein into the interior of the flexible member.

4. The method of claim 2, wherein the glass vial has an elongated, narrow, cylindrical configuration, and depressing the flexible member includes depressing the flexible member to cause a force to be imparted to the breakable, elongated, narrow, cylindrical glass vial to break the vial, releasing the second fluid therein into the interior of the flexible member.

5. The method of claim 1, wherein the fluid transfer and mixing collection assembly further includes a sample tube having a proximal end connected to the inlet and a distal end, and the method further includes communicating the distal end of the sample tube with the first fluid to draw the first fluid into the interior of the flexible member.

6. The method of claim 5, wherein the sample tube is at least one of fixed to the inlet, retractable, not retractable, locked to the inlet, not locked to the inlet, detachably connectable to the inlet.

7. The method of claim 1, wherein the fluid transfer and mixing collection assembly further includes at least one of a wick, sponge, open-cell foam, porous material, and an absorbent material connected to the inlet, and the method further includes communicating at least one of the wick, sponge, open-cell foam, porous material, and an absorbent material with the first fluid to draw the first fluid into the interior of the flexible member.

8. The method of claim 1, wherein the inlet check valve only allows fluid into the flexible member and the outlet check valve only allows fluid out of the flexible member.

9. The method of claim 1, wherein the method is an assay test method, the first fluid is a sample fluid including an analyte of interest for assay testing, the second fluid is a reagent, and the test media visually indicates the presence or absence of an analyte of interest.

10. The method of claim 1, wherein the method is a test method for testing at least one of drug screening, chemical analysis, crime/accident scene investigations, ground water testing (EPA), and livestock testing.

11. The method of claim 1, wherein the first fluid is a fluid medium of at least one of a gas, a liquid, a suspension, an extracted or dissolved sample, and a supercritical fluid.

12. The method of claim 1, wherein the first fluid includes a sample including one or more target analytes of interest for detection.

13. The method of claim 12, wherein the one or more target analytes of interest include at least one of antigen, antibody, receptor, ligands chelate, protein, enzyme, nucleic acid, DNA, RNA, pesticide, herbicide, inorganic compound, organic compounds, a material for which a specific binding reagent exists.

14. The method of claim 1, wherein test media indicates at least one of presence, absence, and concentration of one or more analytes.

15. The method of claim 1, wherein the method is performed by a single hand of the user and by depressing and releasing the flexible member with a thumb of the single hand.

16. The method of claim 1, wherein the vial includes multiple vials including different fluids therein.

17. A fluid transfer and mixing collection assembly, comprising:
a base;
a test media carried by the base;
an inlet for receiving a first fluid, the inlet including an inlet check valve;
an outlet including an outlet check valve;
a breakable vial carried by the base between the inlet and the outlet and including an interior with a second fluid therein, and
a depressable, flexible member carried by the base between the inlet and the outlet, the flexible member including an interior with the breakable vial therein, the flexible member depressable to cause a force to be imparted to the breakable vial to break the vial, releasing the second fluid therein into the interior of the flexible member, the flexible member releasable to impart a negative pressure in the interior of the flexible member to draw the first fluid into the interior of the flexible member through the inlet check valve to mix with the second fluid, and the flexible member depressable again to impart a positive pressure in the interior of the flexible member to pump the mixed first fluid and second fluid out of the interior of the flexible member through the outlet check valve and be transferred to the test media.

18. The fluid transfer and mixing collection assembly of claim 17, wherein the vial is a glass vial, and the flexible member is depressable to cause a force to be imparted to the breakable vial to break the vial, releasing the second fluid therein into the interior of the flexible member.

19. The fluid transfer and mixing collection assembly of claim 17, wherein the glass vial is fixably mounted within the interior of the flexible member, and the flexible member is depressable to cause a force to be imparted to the breakable vial fixably mounted within the interior of the flexible member to break the vial, releasing the second fluid therein into the interior of the flexible member.

20. The fluid transfer and mixing collection assembly of claim 17, wherein the glass vial has an elongated, narrow, cylindrical configuration.
